(19) United States
(12) Patent Application Publication
(10) Pub. No.: US 2007/0221212 A1
(43) Pub. Date: Sep. 27, 2007

(54) Liquid nebulizer

(76) Inventor: Yi-Hsin Huang, Taipei City (TW)

Correspondence Address: HDSL, 4331 STEVENS BATTLE LANE, FAIRFAX, VA, 22033, US

(21) Appl. No.: 11/686,971

(22) Filed: Mar. 16, 2007

(30) Foreign Application Priority Data
Mar. 21, 2006 (TW) 95204676

(51) Int. Cl. A61M 11/00

(52) U.S. Cl. 128/200.16; 239/338; 128/200.14

(57) ABSTRACT

A liquid nebulizer utilizes a channel forming element to position a portion of a sonic vibrator and form a channel passing to a medication cup. When the sonic vibrator vibrates up and down, the liquid from the medication cup passes the channel toward a mesh plate for nebulizing the liquid. The liquid nebulizer provides an easily manufactured channel forming element and has a simple structure. Furthermore, the manufacturing cost of the liquid nebulizer is inexpensive.

### BACKGROUND OF THE INVENTION

1. Field of the Invention

The present invention relates in general to a liquid nebulizer. More particularly, the present invention relates to an ultrasonic liquid nebulizer.

2. Description of the Related Art

Medical nebulizers that nebulize a fluid into a fine spray or aerosol for inhalation by a patient are well-known devices commonly used for the treatment of certain conditions and diseases. Persons requiring treatment of certain kinds of respiratory conditions frequently need to have medications delivered directly to the lungs. Since the lungs are close to the heart and the blood circulatory system of the body, drug administration by inhalation provides an effective delivery system to all organs of the body. Further, medical nebulizers have applications for conscious, spontaneously-breathing patients and for controlled, ventilated patients.

Effective and economical nebulizer therapy includes the ability to quickly generate a large amount of aerosol within a predetermined range of particle size. There are several other considerations that relate to the effectiveness of nebulization therapies. For example, it has been suggested that nebulization therapy is more effective when the generation of aerosol particles is relatively uniform.

With reference to FIG. 6, a liquid nebulizing element of a conventional ultrasonic liquid nebulizer comprises a medication cup (100), a sonic vibrator (105), a mesh plate (110) and a vibrating element (115). The medication cup (100) is used to storage liquid. The sonic vibrator (105) longitudinally comprises a proximal end (105a), a circular vibrating unit (105c, 105d), a tube (105e) and a distal end (105b). The tube (105e) comprises a through channel (105f) and attached with the liquid received in the medication cup (100) through the proximal end (105a) and mounted with the mesh plate (110) through the distal end (105b). The circular vibrating unit (105c, 105d) formed around a portion of the tube (105e). In application, the vibrating element (115) provides a voltage with high frequency to let the circular vibrating unit (105c, 105d) vibrates and induce the tube (105e) vibrates up and down. According to the foregoing description, the liquid received in the medication cup (100) is passed through the proximal end (105a), the through channel (105f) of the tube (105e) and flowing out from the distal end (105b) to the mesh plate (110) into a fine spray.

However, the thin tube of conventional ultrasonic nebulizer is difficult to manufacture and the manufacturing cost is expensive. Furthermore, it is not easy to wash the tube and the through channel of the tube may be blocked easily. Thus, a need exists for improved nebulizer.

### SUMMARY OF THE INVENTION

In order to solve the above noted conventional problems, one aspect of the present invention is to provide a liquid nebulizer that is manufactured easily and cost inexpensively.

One aspect of the present invention is to provide a liquid nebulizer comprising:

a sonic vibrator for generating sonic vibrating;

a channel forming element mounted around the sonic vibrator to form a channel between the channel forming element and the sonic vibrator;

a medication cup mounted around the channel forming element for receiving liquid that is communication with the channel;

a mesh plate mounted on the sonic vibrator and in the channel forming element; and

a sealing element mounted in the channel forming element and between the mesh plate and the channel forming element; wherein

the liquid received in the medication cup will pass through the channel to the mesh plate for nebulization when the sonic vibrator vibrates.

In a preferred embodiment of the present invention, the nebulizer is an ultrasonic liquid nebulizer.

The channel forming element employed in the present invention preferably comprises a hollow tube for mounting a portion of the sonic vibrator to form the channel between the tube and the sonic vibrator. More preferably, the channel forming element further comprises an element proximal end formed on the hollow tube for mounting the sealing element.

In another preferred embodiment of the present invention, the channel forming element comprises a plate for mounting with the sealing element, and furthermore, the plate may have a notch for mounting the sealing element.

Preferably, the medication cup further comprises a side wall to form the channel forming element. In addition, the channel forming element may be a tube mounted around the partial sonic vibrator.

In a preferred embodiment of the present invention, the sealing element further comprises a circular sealing unit mounted around the mesh plate and the circular sealing unit can be composed of an elastic and waterproof material. More preferably, the sealing element further comprises a fastened unit mounted on and around the mesh plate and the mesh plate is more preferably located between the fastened unit and the circular sealing unit.

Preferably, the medication cap employed in the present invention further comprises a cup proximal end and a cup distal end and wherein

the cup proximal end is connected with the channel forming element, and

the cup distal end is connected with the sonic vibrator.

More preferably, the sonic vibrator comprises a main body and a connector formed on the main body and wherein

the main body is a mainly ultrasonic vibrating generation unit which is connected with the cup distal end of the medication cup.

More preferably, the connector is attached with the liquid received in the medication cap and the channel forming element is mounted around the connector of the sonic vibrator and connected with the cup proximal end of the medication cup.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a side view of an ultrasonic liquid nebulizer in accordance with the present invention;

FIG. 2 is a partial cross-sectional view of a preferred embodiment showing A-A line of FIG. 1;

FIG. 3 is a partial cross-sectional view of another preferred embodiment showing A-A line of FIG. 1;

FIG. 4 is a partial cross-sectional view of a further preferred embodiment showing A-A line of FIG. 1;

FIGS. 5A, 5B, 5C, 5D are cross-sectional views respectively showing a location of a channel, a channel forming element and a connector of the nebulizer in accordance with the present invention; and

FIG. 6 is a perspective view of a liquid nebulizing element of a conventional ultrasonic liquid nebulizer.

### DESCRIPTION OF THE PREFERRED EMBODIMENTS

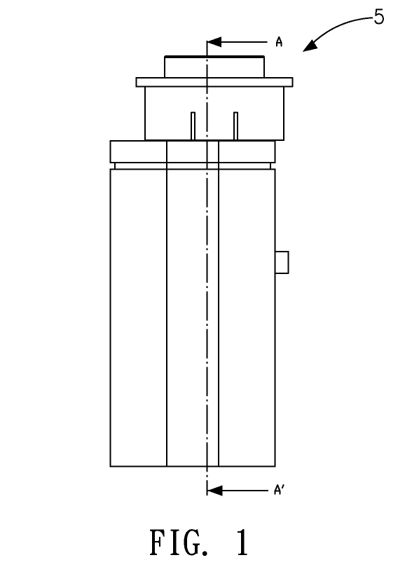

With reference to FIG. 1, it is a side view showing a preferred embodiment of an ultrasonic liquid nebulizer (5) in accordance with the present invention. The ultrasonic liquid nebulizer (5) can be like, but is not limited to, a circular column.

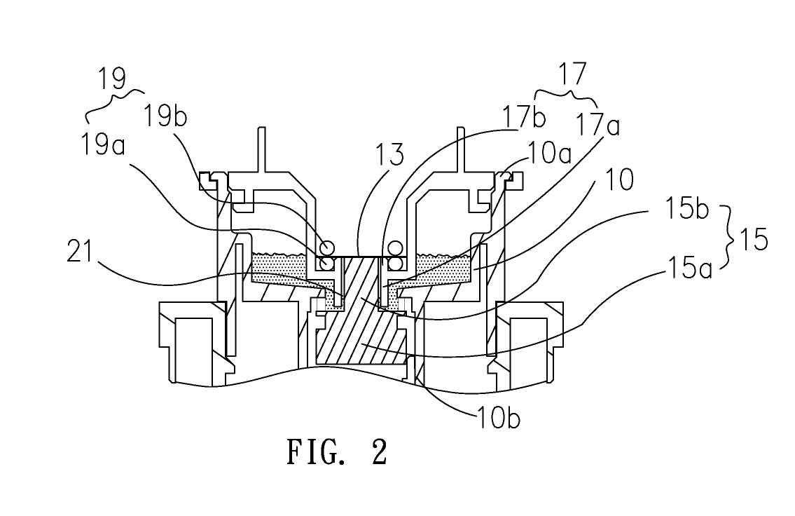

With reference to FIG. 2, it is a partial cross-sectional view of a preferred embodiment showing A-A line of FIG. 1. The ultrasonic liquid nebulizer (5) comprises a medication cup (10), a sonic vibrator (15), a mesh plate (13), a channel forming element (17) and a sealing element (19).

The medication cap (10) is used for receiving liquid and comprises a cup proximal end (10a) and a cup distal end (10b). The sonic vibrator (15) is mounted in the medication cup (10) and connected with the cup distal end (10b) of the medication cup (10) and comprises longitudinally a main body (15a) and a connector (15b) formed on the main body (15a). In a preferred embodiment, the main body (15a) such as a vibrator is a mainly ultrasonic vibrating generation unit which is connected with the cup distal end (10b) of the medication cup (10) and the connector (15b) vibrates up and down with the main body (15a). Further, the connector (15b) is attached with the liquid received in the medication cap (10) and the channel forming element (17) is mounted around the connector (15b) of the sonic vibrator (15) and connected with the cup proximal end (10a) of the medication cup (10).

In a preferred embodiment of the present invention, the channel forming element (17) is a hollow tube and comprises a body (17a), an element proximal end (17b) formed on the body (17a) and an inner wall, thereby the connector (15b) can be mounted in the hollow tube and leave a thin channel (21) between the inner wall of the channel forming element (17) and the connector (15b) for letting the liquid received in the medication cup (10) passed from the medication cup (10) to the channel (21). Preferably, the liquid received in the medication cup (10) can climb toward to a top of the connector (15b) by the action of the ultrasonic vibrating when the sonic vibrator (15) produces ultrasonic vibrating. Preferably, the channel (21) just provides the space for the liquid to climb up so the channel (21) only needs an enough thin path as fine and do not need to couple the shape with the body (17a) of the channel forming element (17) and the connector (15b). Of course, the channel (21) has an equal diameter is preferred but is not limited to.

Preferably, the element proximal end (17b) of the channel forming element (17) is used for setting the sealing element (19) and the mesh plate (13). Further, the connector (15b) of the sonic vibrator (15) is extended higher than the element proximal end (17b) of the channel forming element (17). In a preferred embodiment, the element proximal end (17b) of the channel forming element (17) comprises a plate extended perpendicularly from the element proximal end (17b) for receiving the sealing element (19). The sealing element (19) is preferably formed by a waterproof material and/or silica gel and formed a circular sealing unit (19a) to prevent the liquid from leaking out through the around of the element proximal end (17b) of the channel forming element (17). In addition, the circular sealing unit (19a) can change shape appropriately when the connector (15b) vibrates up and down, for example, the circular sealing unit (19a) is composed of an elastic material. Preferably, the body (17a) of the channel forming element (17) further comprises an extending unit (not shown) extended from the element proximal end (17b) for forming a notch for mounting the circular sealing unit (19a) and is not limited to the foregoing plate shape.

The mesh plate (13) is mounted on the circular sealing unit (19a) and coupled with the element proximal end (17b) of the channel forming element (17). In a preferred embodiment, the liquid climbed through the channel (21) is guided by the circular sealing unit (19a) to the mesh plate (13) for nebulization outside. Preferably, the sealing element (19) further comprises a fastened unit (19b) mounted on and around the mesh plate (13) to fasten the mesh plate (13) and enhance the sealing function. The shape and the composed material of the fastened unit (19b) can be, but is not limited to, the same with the circular sealing unit (19a).

According to the foregoing description, the mesh plate (13), the sealing element (19) and the channel forming element (17) in accordance with the present invention have simple structure and are mounted easily, washed conveniently and cost cheaply.

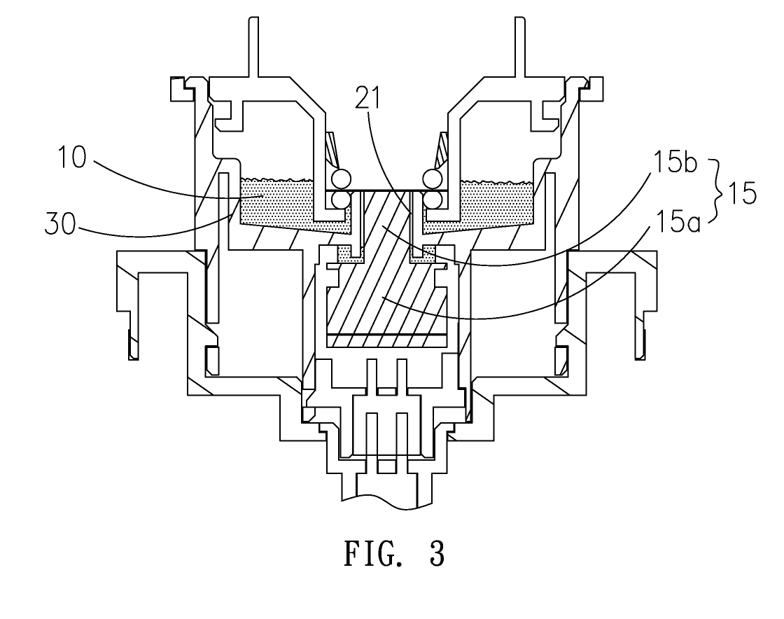

With further reference to FIG. 3, it is another preferred embodiment showing a new structure of a medication cup (30) that has a side wall coupled with the connector (15b) to form the channel (21). Preferably, the side wall of the medication cup (30) has a plurality of openings or holes for the liquid to pass through.

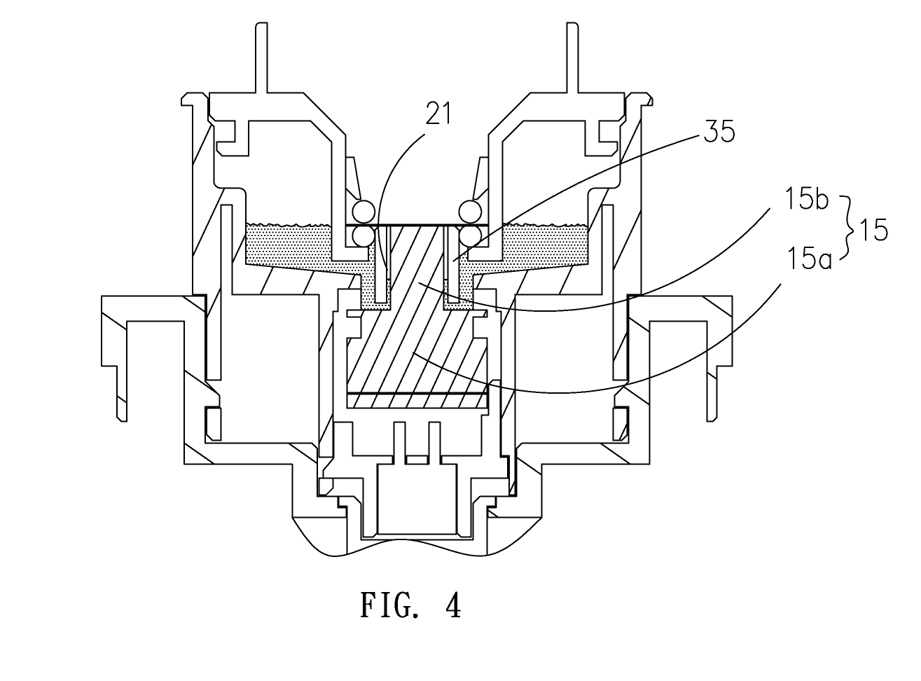

With further reference to FIG. 4, it is another preferred embodiment showing another structure to form the channel (21). The nebulizer further comprises an independent tube (35) mounted around the connector (15b) of the sonic vibrator (15) to form the channel (21) between the independent tube (35) and the connector (15b).

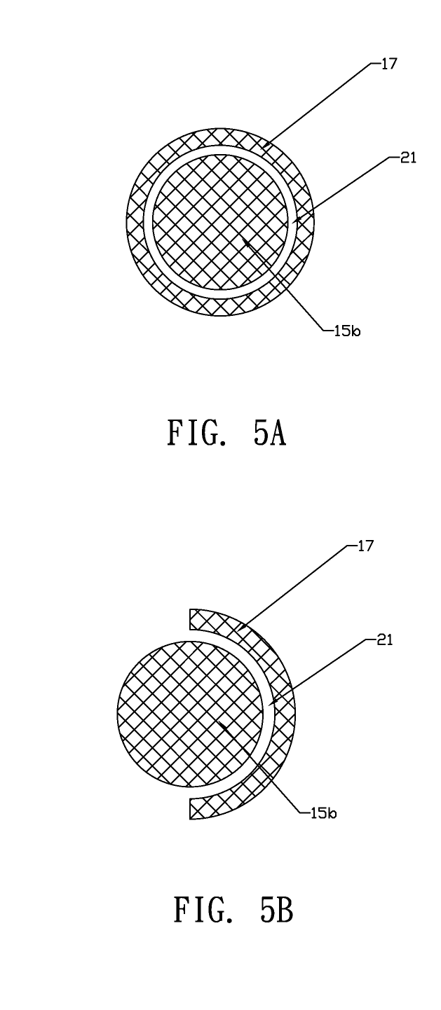

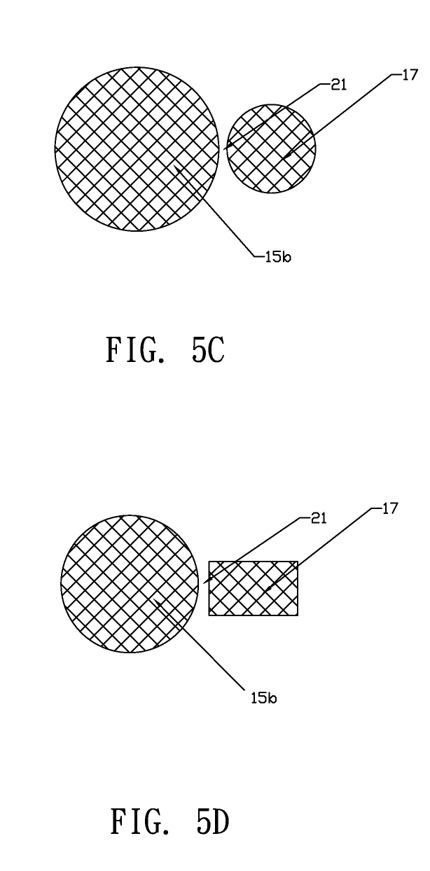

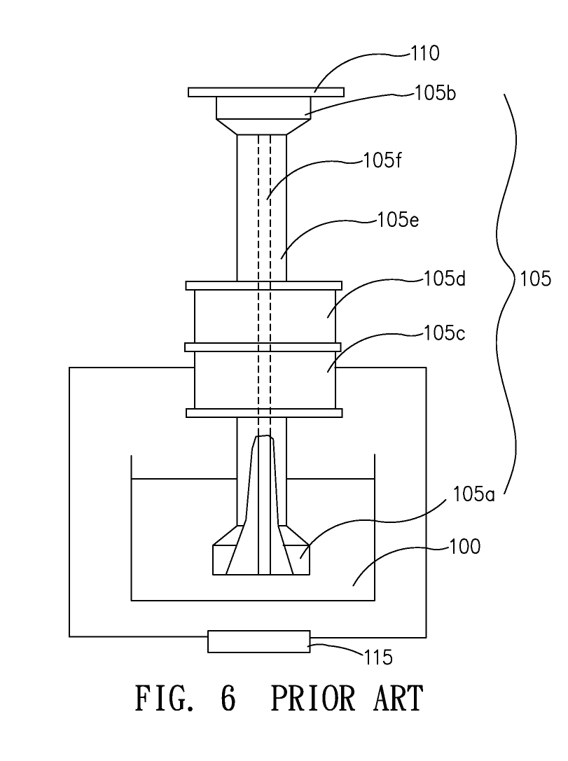

With reference to FIGS. 5A to 5D, they are showing different location between the channel (21), the connector (15b) and the channel forming unit (17). According to the spirit of the present invention, the shape of the channel forming element (17) and the connector (15b) has no limit for its shape, such as circle, semi-circle, circular column or square column thus the channel (21) also has no limit for its shape.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. A liquid nebulizer comprising:
a sonic vibrator for generating sonic vibrating;
a channel forming element mounted around the sonic vibrator to form a channel between the channel forming element and the sonic vibrator;
a medication cup mounted around the channel forming element for receiving liquid that is communication with the channel;
a mesh plate mounted on the sonic vibrator and in the channel forming element; and
a sealing element mounted in the channel forming element and between the mesh plate and the channel forming element; wherein
the liquid received in the medication cup will pass through the channel to the mesh plate for nebulization when the sonic vibrator vibrates.

2. The nebulizer as claimed in claim 1, wherein the channel forming element comprises a hollow tube for mounting a portion of the sonic vibrator to form the channel between the tube and the sonic vibrator.

3. The nebulizer as claimed in claim 1, wherein the channel forming element further comprises an element proximal end formed on the hollow tube for mounting the sealing element.

4. The nebulizer as claimed in claim 1, wherein the sealing element further comprises a circular sealing unit mounted around the mesh plate.

5. The nebulizer as claimed in claim 4, wherein the circular sealing unit is composed of an elastic and waterproof material.

6. The nebulizer as claimed in claim 4, wherein the sealing element further comprises a fastened unit mounted on and around the mesh plate.

7. The nebulizer as claimed in claim 6, wherein the mesh plate is located between the fastened unit and the circular sealing unit.

8. The nebulizer as claimed in claim 1, wherein the medication cup further comprises a side wall to form the channel forming element.

9. The nebulizer as claimed in claim 1, wherein the channel forming element is a tube mounted around the partial sonic vibrator.

10. The nebulizer as claimed in claim 1, wherein the nebulizer is an ultrasonic liquid nebulizer.

11. The nebulizer as claimed in claim 1, wherein the channel forming element comprises a plate for mounting with the sealing element.

12. The nebulizer as claimed in claim 11, wherein the plate has a notch for mounting the sealing element.

13. The nebulizer as claimed in claim 1, wherein the medication cap further comprises a cup proximal end and a cup distal end and wherein
the cup proximal end is connected with the channel forming element, and
the cup distal end is connected with the sonic vibrator.

14. The nebulizer as claimed in claim 13, wherein the sonic vibrator comprises a main body and a connector formed on the main body and wherein
the main body is a mainly ultrasonic vibrating generation unit which is connected with the cup distal end of the medication cup.

15. The nebulizer as claimed in claim 14, wherein the connector is attached with the liquid received in the medication cap and the channel forming element is mounted around the connector of the sonic vibrator and connected with the cup proximal end of the medication cup.
